(19) United States
(12) Patent Application Publication
(10) Pub. No.: US 2021/0059396 A1
(43) Pub. Date: Mar. 4, 2021

(54) HANDHELD MULTIFUNCTIONAL FACIAL CLEANSING BRUSH STRUCTURE

(71) Applicant: YIQUN TANG, JI'AN (CN)

(72) Inventor: YIQUN TANG, JI'AN (CN)

(21) Appl. No.: 17/095,770

(22) Filed: Nov. 12, 2020

(30) Foreign Application Priority Data
Nov. 20, 2019 (CA) 201922010098.X

(51) Int. Cl. A46B 15/00; H01M 10/052; H01M 10/46; A46B 5/02; H05B 1/02; A61H 7/00; A61H 23/02; A61N 5/06

(52) U.S. Cl. CPC A46B 15/0022; A46B 15/0036; A46B 15/0034; A46B 15/003; H01M 10/052; A46B 15/0075; H02J 7/0045; A46B 5/026; H05B 1/025; A61H 7/005; A61H 23/02; A61N 5/0616; H01M 10/46

(57) ABSTRACT

The present invention provides a handheld multifunctional facial cleansing brush structure, including a housing, a power supply assembly, a control switch, a massage assembly, a thermal conduction assembly, and a silicone sleeve. The power supply assembly, the control switch, the massage assembly, and the thermal conduction assembly are electrically connected to the power supply assembly. The thermal conduction assembly passes through an upper surface of the housing to form a thermal conduction working face. The massage assembly passes through a lower surface of the housing to form a massage working face. The housing is covered by the silicone sleeve. The present invention has a thermal conduction massage function and an LED phototherapy function and is convenient to use. In addition, a surrounding design of massage bumps better fits with the face and provides a more comfortable massage.

### TECHNICAL FIELD

The present invention relates to the technical field of daily necessities, and in particular, to a handheld multifunctional facial cleansing brush structure.

### BACKGROUND

With the development of economy and the changing of social conditions, people pay increasing attention to their appearance. Facial cleansing brushes appear as science and technology advance. A facial cleansing brush is a readily operable and portable device that comforts and cleanses the skin, and helps the skin to absorb skin care products based on physical therapy.

At present, there are various facial cleansing brushes providing a variety of facial cleansing methods. A single facial cleansing instrument uses a single method to cleanse the face, but this method can only clean a single part of the face, and cannot change the cleansing part according to needs, so the cost performance is extremely low. Even if the cleansing parts can be changed, the cleansing effect of the facial cleansing brush cannot be ensured.

In the prior art, a facial cleansing brush uses various facial cleansing heads to achieve the cleansing of various parts of the face, which improves the cost performance. However, it is inconvenient to carry and change the various facial cleansing heads, and the facial cleansing heads are prone to loss. In addition, a single facial cleansing head, when used to cleanse the face, provides poor cleansing effect and poor comfort. Generally, the facial cleansing brush has only a cleansing function and a massage function. After the skin is cleansed, an additional skin phototherapy instrument is required for the phototherapy of skin. Therefore, there is a need for a multifunctional handheld facial cleansing brush to resolve the foregoing problems.

### SUMMARY

The present invention provides a handheld multifunctional facial cleansing brush structure, which resolves the problems of single function and poor comfort of an existing facial cleansing brush by making technical improvement thereto.

To resolve the foregoing technical problems, the present invention specifically adopts the following technical solution.

A handheld multifunctional facial cleansing brush structure, including a housing, a power supply assembly, a control switch, a massage assembly, a thermal conduction assembly, and a silicone sleeve, the power supply assembly, the control switch, the massage assembly, and the thermal conduction assembly being electrically connected to the power supply assembly, the thermal conduction assembly passing through an upper surface of the housing to form a thermal conduction working face, the massage assembly passing through a lower surface of the housing to form a massage working face, and the housing being covered by the silicone sleeve.

Preferably, the power supply assembly includes a magnetic charging connector and a lithium battery, the lithium battery is fixedly disposed inside the housing, the magnetic charging connector passes through the silicone sleeve and the housing to be electrically connected to the lithium battery, and the lithium battery can be connected to an external power supply by the magnetic charging connector.

Preferably, the thermal conduction assembly includes a heat generation sheet and an alloy disk, the alloy disk passes through the housing to be exposed to the outside, a bottom surface of the alloy disk is provided with the heat generation sheet, the heat generation sheet is electrically connected to the power supply assembly, and the alloy disk is circumferentially provided with an LED assembly.

Preferably, the LED assembly includes an annular LED lamp group which includes a red LED lamp, a blue LED lamp and a green LED lamp and has a wavelength of 400 nm to 700 nm.

Preferably, the massage assembly includes a vibration motor, a mounting groove is provided in the back of the massage working face inside the housing, the vibration motor is fixedly disposed inside the mounting groove and is electrically connected to a power supply module, and a plurality of protruding massage bumps are formed on the silicone sleeve outside the housing corresponding to the massage working face.

Preferably, a cavity is provided between the silicone sleeve and the massage working face, a silicone support post is provided inside the cavity to abut against the housing, and the massage working face of the housing is provided with vent holes.

Compared with the prior art, the present invention has the following beneficial effects:
(1) According to the present invention, the hollow cavity is provided inside the silicone sleeve, the vent holes are arranged in the housing, and the silicone support post is provided between the silicone sleeve and the housing. The provision of the vent holes in the housing enables a negative pressure to be formed during the use of the surrounding massage bumps to provide cushioning and resilience, thereby improving the comfort of face cleansing and massage.
(2) According to the present invention, the LED assembly is provided, so that LED illumination of specific wavelengths are provided to produce a phototherapeutic effect of skin care and beautification.
(3) According to the present invention, one face is provided with the thermal conduction working face by means of the thermal conduction assembly, and the other face is provided with the massage working face, so that a small volume is occupied, versatile functions are provided, and the use is convenient.

### BRIEF DESCRIPTION OF THE DRAWINGS

FIG. 1 is a schematic diagram of a thermal conduction working face according to the present invention;

FIG. 2 is a schematic diagram of a massage working face according to the present invention;

FIG. 3 is a schematic exploded view according to the present invention;

FIG. 4 is a schematic diagram of a massage working face when the silicone sleeve is sleeved according to the present invention;

FIG. 5 is a schematic diagram of a thermal conduction working face when the silicone sleeve is sleeved according to the present invention; and

FIG. 6 is a schematic diagram of the interior of the silicone sleeve according to the present invention.

Reference numerals: housing 1, waterproof silicone pad 11, mounting groove 12, vent hole 13, power supply assembly 2, magnetic charging connector 21, lithium battery 22, control switch 3, massage assembly 4, vibration motor 41, thermal conduction assembly 5, heat generation sheet 51, alloy disk 52, silicone sleeve 6, massage bump 61, silicone support post 62, concave groove 63, lug 64, thermal conduction working face 7, massage working face 8, and LED assembly 9.

### DETAILED DESCRIPTION

Specific content of the present invention is described below in detail with reference to the accompanying drawings and the embodiments.

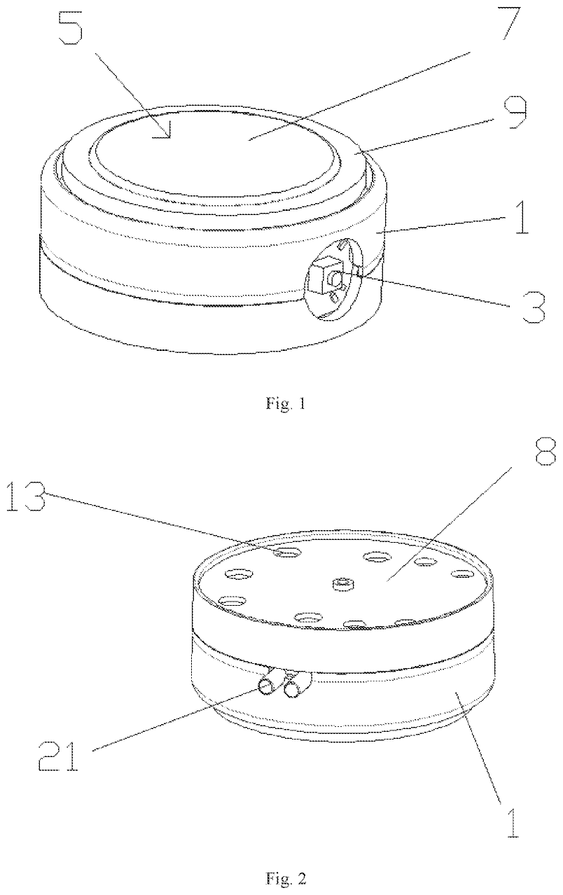

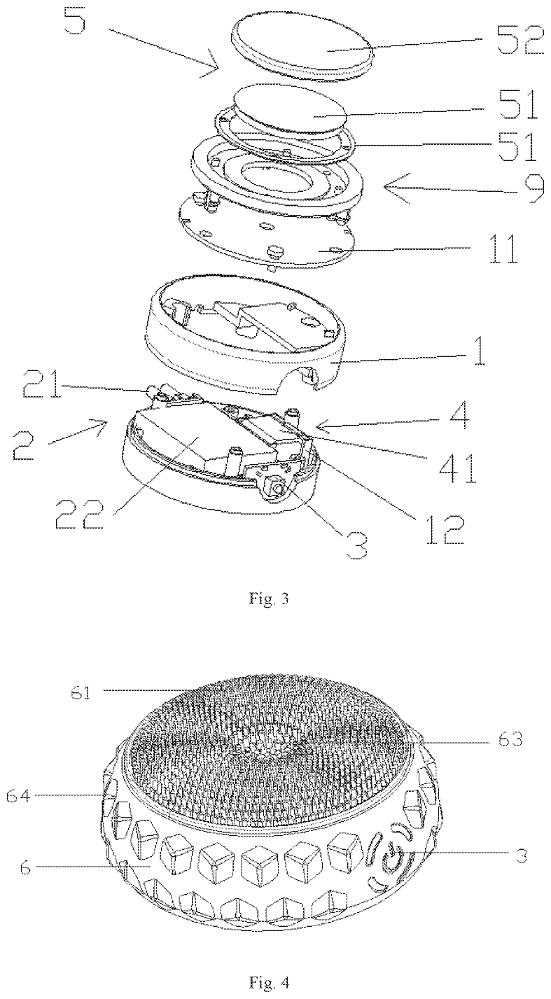

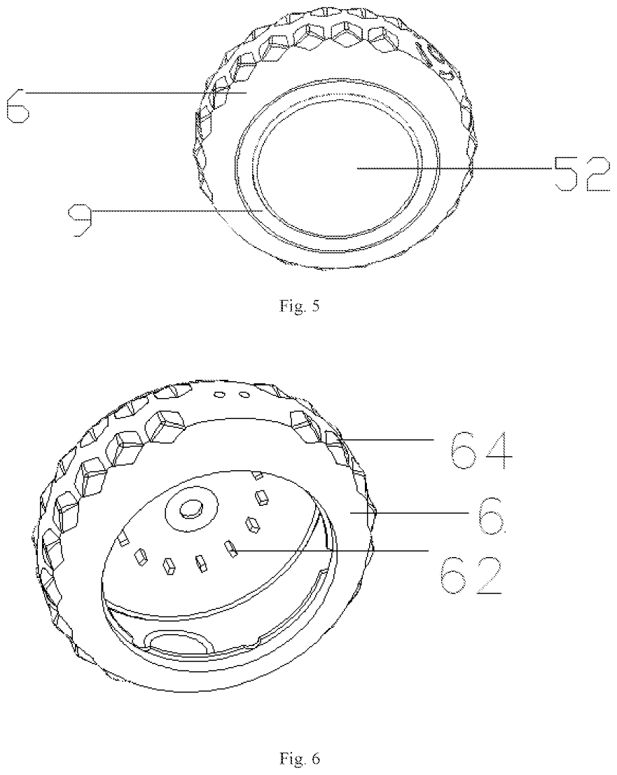

As shown in FIG. 1 to FIG. 6, Embodiment 1 provides a handheld multifunctional facial cleansing brush structure, including a housing 1, a power supply assembly 2, a control switch 3, a massage assembly 4, a thermal conduction assembly 5, and a silicone sleeve 6. The power supply assembly 2, the control switch 3, the massage assembly 4, and the thermal conduction assembly 5 are electrically connected to the power supply assembly 2. The thermal conduction assembly 5 passes through an upper surface of the housing 1 to form a thermal conduction working face 7. The massage assembly 4 passes through a lower surface of the housing 1 to form a massage working face 8. The housing 1 is covered by the silicone sleeve 6.

Further, to enable the power supply assembly 2 to supply power to the parts, the power supply assembly 2 includes a magnetic charging connector 21 and a lithium battery 22, the lithium battery 22 is fixedly disposed inside the housing 1, the magnetic charging connector 21 passes through the silicone sleeve 6 and the housing 1 to be electrically connected to the lithium battery 22, and the lithium battery 22 can be connected to an external power supply by the magnetic charging connector 21.

Further, to implement the thermal conduction function of the thermal conduction assembly 5, the thermal conduction assembly 5 includes a heat generation sheet 51 and an alloy disk 52, the alloy disk 52 passes through the housing 1 to be exposed to the outside, a bottom surface of the alloy disk 52 is provided with the heat generation sheet 51, the heat generation sheet 51 is electrically connected to the power supply assembly 2, and the alloy disk 52 is circumferentially provided with an LED assembly 9. The heat generation sheet 51 keeps a surface temperature of the alloy disk 52 between 40 degrees and 55 degrees to provide thermal conduction for massage and thermal conduction for facial cleansing cream, skin care products or facial masks. A temperature control system may further be incorporated to the thermal conduction assembly 5.

Further, to achieve a better waterproof effect, a waterproof silicone pad 11 is further provided inside the housing 1, and the waterproof silicone pad 11 is located below the thermal conduction assembly 5 and the LED assembly 9 and above the power supply assembly 2 and a vibration LED assembly 9.

Further, to enable the LED assembly 9 to achieve a better phototherapeutic effect, the LED assembly 9 includes an annular LED lamp group which includes a red LED lamp, a blue LED lamp and a green LED lamp and has a wavelength of 400 nm to 700 nm. The illumination with light of a wavelength between 400 nm and 700 nm can provide the effect of skin care and beautification.

Further, to achieve a better vibration massage effect, the massage assembly 4 includes a vibration motor 41, a mounting groove 12 is provided in the back of the massage working face 8 inside the housing 1, the vibration motor 41 is fixedly disposed inside the mounting groove 12 and is electrically connected to a power supply module, and a plurality of protruding massage bumps 61 are formed on the silicone sleeve 6 outside the housing 1 corresponding to the massage working face 8.

Further, to achieve a cushioning and resilience effect of massage and improve the level of comfort for face cleansing and massage, a cavity is provided between the silicone sleeve 6 and the massage working face 8, a silicone support post 62 is provided inside the cavity to abut against the housing 1, and the massage working face 8 of the housing 1 is provided with vent holes 13.

Further, to achieve a more comfortable facial cleansing effect, the massage bumps 61 are distributed in an arc form and rise and fall to form a concave groove 63 by concaving from an outer ring towards the center.

Further, to achieve better grip and prevent slippage during holding, an outer side wall of the silicone sleeve 6 is further evenly provided with a plurality of lugs 64.

According to this embodiment, the hollow cavity is provided inside the silicone sleeve 6, the vent holes 13 are arranged in the housing 1, and the silicone support post 62 is provided between the silicone sleeve 6 and the housing 1. The provision of the vent holes 13 in the housing 1 enables a negative pressure to be formed during the use of the surrounding massage bumps 61 to provide cushioning and resilience, thereby improving the comfort of face cleansing and massage.

According to this embodiment, the LED assembly 9 is provided, so that LED illumination of specific wavelengths are provided to produce a phototherapeutic effect of skin care and beautification. In this embodiment, one face is provided with the thermal conduction working face 7 by means of the thermal conduction assembly 5, and the other face is provided with the massage working face 8, so that a small volume is occupied, versatile functions are provided, and the use is convenient.

In another Embodiment 2, the massage bumps are arranged on the massage working face in a hemispherical manner, and the center is higher than the periphery. During use, the facial cleansing brush may be held to make the massage bumps touch the face. Through rolling massage, a massage contact area is reduced, the massage strength is higher, and a better massage effect is achieved, thus providing a different massage manner.

In another Embodiment 3, the massage bumps are divided into a dense area and a sparse area. In the dense area, the massage bumps have small gaps and are densely distributed, while in the sparse area, the massage bumps have large gaps and are sparsely distributed. Different combined textures of the massage bumps are applicable to people with various skin types, for example, sensitive skin, oily skin, and normal skin.

In another Embodiment 4, the power supply assembly includes a cable and an external plug, to facilitate long-time home use and provide more stable power supply.

In another Embodiment 5, an outer side wall of the silicone sleeve is provided with anti-slip patterns or anti-slip raised lines, to avoid slippage and provide more comfortable grip.

Finally, it should be noted that the foregoing embodiments are merely intended for describing the technical solutions of the present invention rather than limiting the present invention. Although the present invention is described in detail with reference to the preferred embodiments, persons of ordinary skill in the art should understand that they may still make modifications or equivalent replacements to the technical features of the present invention without departing from the objective and scope of the technical solutions of the technical solutions of the present invention. These modifications or equivalent replacements shall all fall within the scope of the claims the present invention.

The standard parts used in the present invention are all commercially available, and the irregular parts can be customized according to the description and drawings. Mature conventional methods such as bolts, rivets, and welding in the prior art are adopted for the specific connection methods of all parts. Conventional models in the prior art are adopted for all the machines, parts, and equipment. Conventional connection methods in the prior art are adopted for circuit connection. Details are not described herein again in detail.

In the description of the present invention, unless otherwise expressly specified and defined, “mounted”, “connected”, “connection”, and “fixed” should be understood in a broad sense, for example, fixedly connected, detachably connected or integrally connected; or mechanically connected or electrically connected; or connected directly or through an intermediate, or two elements communicated internally or two elements having mutual effects. For persons skilled in the art, specific meanings of the terms in the present invention should be understood according to specific conditions.

1. A handheld multifunctional facial cleansing brush structure, comprising a housing, a power supply assembly, a control switch, a massage assembly, a thermal conduction assembly, and a silicone sleeve, the power supply assembly, the control switch, the massage assembly, and the thermal conduction assembly being electrically connected to the power supply assembly, the thermal conduction assembly passing through an upper surface of the housing to form a thermal conduction working face, the massage assembly passing through a lower surface of the housing to form a massage working face, and the housing being covered by the silicone sleeve.

2. The handheld multifunctional facial cleansing brush structure according to claim 1, wherein the power supply assembly comprises a magnetic charging connector and a lithium battery, the lithium battery is fixedly disposed inside the housing, the magnetic charging connector passes through the silicone sleeve and the housing to be electrically connected to the lithium battery, and the lithium battery can be connected to an external power supply by the magnetic charging connector.

3. The handheld multifunctional facial cleansing brush structure according to claim 1, wherein the thermal conduction assembly comprises a heat generation sheet and an alloy disk, the alloy disk passes through the housing to be exposed to the outside, a bottom surface of the alloy disk is provided with the heat generation sheet, the heat generation sheet is electrically connected to the power supply assembly, and the alloy disk is circumferentially provided with an LED assembly.

4. The handheld multifunctional facial cleansing brush structure according to claim 3, wherein a waterproof silicone pad is further provided inside the housing, the waterproof silicone pad is located below the thermal conduction assembly and the LED assembly and above the power supply assembly and a vibration assembly.

5. The handheld multifunctional facial cleansing brush structure according to claim 3, wherein the LED assembly comprises an annular LED lamp group which comprises a red LED lamp, a blue LED lamp and a green LED lamp and has a wavelength of 400 nm to 700 nm.

6. The handheld multifunctional facial cleansing brush structure according to claim 1, wherein the massage assembly comprises a vibration motor, a mounting groove is provided in the back of the massage working face inside the housing, the vibration motor is fixedly disposed inside the mounting groove and is electrically connected to a power supply module, and a plurality of protruding massage bumps are formed on the silicone sleeve outside the housing corresponding to the massage working face.

7. The handheld multifunctional facial cleansing brush structure according to claim 6, wherein a cavity is provided between the silicone sleeve and the massage working face, a silicone support post is provided inside the cavity to abut against the housing, and the massage working face of the housing is provided with vent holes.

8. The handheld multifunctional facial cleansing brush structure according to claim 6, wherein the massage bumps are distributed in an arc form, and rise and fall to form a concave groove by concaving from an outer ring towards the center.

9. The handheld multifunctional facial cleansing brush structure according to claim 1, wherein an outer side wall of the silicone sleeve is further evenly provided with a plurality of lugs.
